(19) United States
(12) Patent Application Publication
(10) Pub. No.: US 2015/0038843 A1
(43) Pub. Date: Feb. 5, 2015

(54) METHOD OF TRANSURETHRAL LOCAL ANESTHESIA

(71) Applicant: Olympus Medical Systems Corp., Tokyo (JP)

(72) Inventor: Kenichi NISHINA, Tokyo (JP)

(21) Appl. No.: 13/958,732

(22) Filed: Aug. 5, 2013

(51) Int. Cl. A61B 17/34; A61M 25/01; A61B 17/00; A61B 8/12; A61B 8/00; A61B 1/307; A61M 25/00; A61B 1/018

(52) U.S. Cl. CPC A61B 17/3478; A61M 25/0084; A61M 25/0105; A61B 1/018; A61B 8/12; A61B 8/445; A61B 1/307; A61B 17/00234; A61M 2202/048; A61M 2210/1089; A61M 2210/1085; A61M 2025/0166; A61B 2017/0034
USPC 600/439

(57) ABSTRACT

A method of transurethral local anesthesia includes: a step I of inserting a flexible ultrasound endoscope having a treatment instrument channel into a urethra or a urinary bladder; a step II of identifying a region to be anesthetized by ultrasound observation using the ultrasound endoscope; a step III of leading an injection needle out from the treatment instrument channel and injecting an anesthetic drug into the region to be anesthetized while performing ultrasound observation using the ultrasound endoscope; a step IV of extracting the ultrasound endoscope from the urethra; and a step V of inserting, after the step IV, an endoscope and/or a treatment instrument into the urethra or the urinary bladder and performing transurethral treatment.

### BACKGROUND OF THE INVENTION

1. Field of the Invention

The present invention relates to a method of transurethral local anesthesia performed before treatment, when the treatment for diagnosis or therapy is performed by inserting an endoscope and/or a treatment instrument from an external urethral orifice into a urethra or a urinary bladder and using the endoscope and/or the treatment instrument.

2. Description of Related Art

As a medical procedure for performing treatment such as diagnosis, therapy, and the like, on a prostate gland, a urinary bladder, or the like, a transurethral medical procedure is known in which an endoscope and/or a treatment instrument is inserted from an external urethral orifice into a urethra or a urinary bladder and treatment is performed on the prostate gland, the urethra, or the like using the endoscope and/or the treatment instrument.

For example, U.S. Patent Publication No. 2004/0002647 discloses in the specification a medical procedure for leading a hollow needle out from a rigid or flexible endoscope inserted into a urethra to puncture the needle into a prostate gland and injecting a treatment substance into the prostate gland. In addition, U.S. Patent Publication No. 2012/0158102 discloses in the specification a medical procedure for resecting a tissue of a prostate gland by using a probe inserted into a urethra.

When such treatment for diagnosis or therapy with the endoscope and/or the treatment instrument inserted into the urethra from the external urethral orifice is performed, there is a case where lumbar anesthesia is performed by sticking a needle from the back of a subject to be examined into a target site in a body, in order to dull the pain felt by the subject to be examined, which is caused by an operation for pressing the mucosa in the urethra or operation for expanding the urethral sphincter performed by using the endoscope and/or the treatment instrument.

### SUMMARY OF THE INVENTION

A method of transurethral local anesthesia according to one aspect of the present invention comprises steps of: a step I of inserting a flexible ultrasound endoscope having a treatment instrument channel into a urethra or a urinary bladder; a step II of identifying a region to be anesthetized by ultrasound observation using the ultrasound endoscope; a step III of leading an injection needle out from the treatment instrument channel of the ultrasound endoscope and injecting an anesthetic drug into the region to be anesthetized while performing ultrasound observation using the ultrasound endoscope; a step IV of extracting the ultrasound endoscope from the urethra; and a step V of inserting, after the step IV, an endoscope and/or a treatment instrument into the urethra or the urinary bladder and performing transurethral treatment.

### BRIEF DESCRIPTION OF THE DRAWINGS

FIG. 1 is a flowchart showing a treatment method according to the present invention.

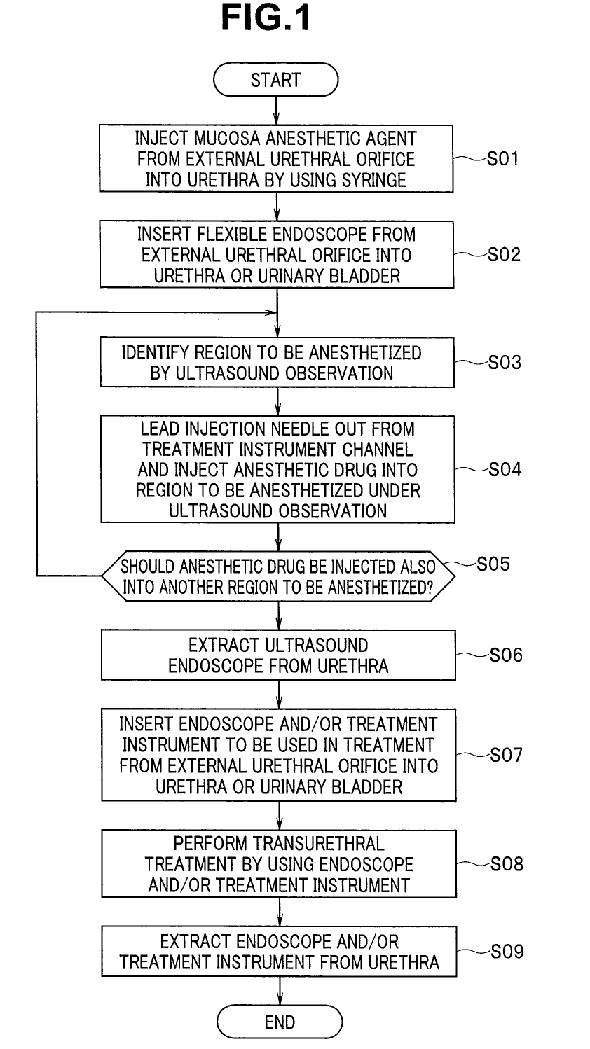

FIG. 2 illustrates prostatic biopsy treatment as an example of transurethral treatment.

FIG. 3 illustrates an example of a configuration of a flexible ultrasound endoscope.

FIG. 4 illustrates a state where an insertion portion of a flexible ultrasound endoscope is inserted from an external urethral orifice into a urethra.

FIG. 5 illustrates a state where the distal end portion of the insertion portion is inserted into a position of a prostate gland.

FIG. 6 illustrates positions of three regions to be anesthetized.

FIG. 7 illustrates a state where a first anesthesia region is set within a scanning region of the ultrasound transducer.

FIG. 8 illustrates a state where an injection needle is punctured into a first region to be anesthetized through the prostate gland.

FIG. 9 illustrates a state where the injection needle is punctured into the first anesthesia region from inside of the urinary bladder.

FIG. 10 illustrates a state where the injection needle is punctured into a second region to be anesthetized through the prostate gland.

FIG. 11 illustrates a state where the injection needle is punctured into the second region to be anesthetized from inside of the urinary bladder.

FIG. 12 illustrates a state where the injection needle is punctured into a third region to be anesthetized.

### DETAILED DESCRIPTION OF PREFERRED EMBODIMENT

Hereinafter, a preferred embodiment of the present invention will be described with reference to drawings. Note that, in the drawings used for the description below, a different scale size is used for each of the components in order to allow each of the components to be illustrated in a recognizable size in the drawings, and the present invention is not limited to the number, shapes, ratio of the sizes of the components, and a relative positional relationship among the components shown in these drawings.

A method of transurethral local anesthesia according to the present invention relates to a method of applying local anesthesia treatment to a subject to be examined before performing treatment, when the treatment for diagnosis or therapy is performed by inserting an instrument from an external urethral orifice into a urethra and using the instrument.

The contents of the transurethral treatment performed after the method of transurethral local anesthesia according to the present invention are not specifically limited.

For example, the transurethral treatment performed on the prostate gland as a region to be treated includes prostatic biopsy treatment for harvesting a tissue of the prostate gland for making a diagnosis, therapeutic treatment for a prostate cancer or a prostatic hyperplasia, or the like. The therapeutic treatment for the prostate cancer includes treatment for injecting ethanol or anticancer drug, radiofrequency ablation, cryotherapy, brachytherapy, etc., for example. In addition, therapeutic treatment for the prostatic hyperplasia includes treatment for resecting a part of the prostate gland, or treatment for heating the prostate gland by a microwave, for example.

In addition, the transurethral treatment performed on the urinary bladder as a region to be treated includes therapeutic treatment for a urinary bladder cancer, for example. The therapeutic treatment for the urinary bladder cancer includes endoscopic resection, radiofrequency ablation, cryotherapy, etc., for example.

Furthermore, for example, the transurethral treatment performed on the urethra or around the urethra as a region to be treated includes therapeutic treatment for a urinary incontinence. The therapeutic treatment for the urinary incontinence includes treatment for injecting collagen into the peripheral region of the urethra.

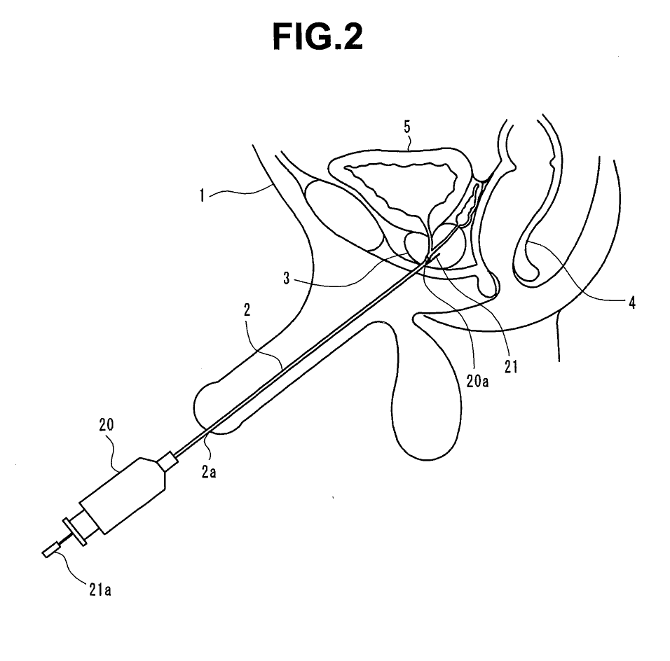

In the present embodiment, the transurethral treatment is supposed to be prostatic biopsy treatment as an example. As shown in FIG. 2, in the prostatic biopsy treatment, a rigid endoscope 20 is inserted from an external urethral orifice 2a into a urethra 2, a biopsy needle 21 is led out from a distal end portion 20a of the rigid endoscope 20, and the biopsy needle 21 is punctured into a prostate gland 3. A tissue of the prostate gland 3 is harvested by puncturing the biopsy needle 21 into the prostate gland 3. A process of puncturing the biopsy needle 21 into the prostate gland 3 is performed under ultrasound observation using an ultrasound observation apparatus, not shown, disposed on a body surface or in a rectum 4 of a subject to be examined 1, for example. Note that the ultrasound observation apparatus may be disposed in the distal end portion 20a of the rigid endoscope 20. The insertion depth of the biopsy needle 21 is adjusted by operating a biopsy needle grasping portion 21a connected to the proximal end of the biopsy needle 21 and exposed from the rigid endoscope 20.

Hereinafter, description will be made on the method of transurethral local anesthesia according to the present embodiment which is performed before the transurethral treatment such as the prostatic biopsy.

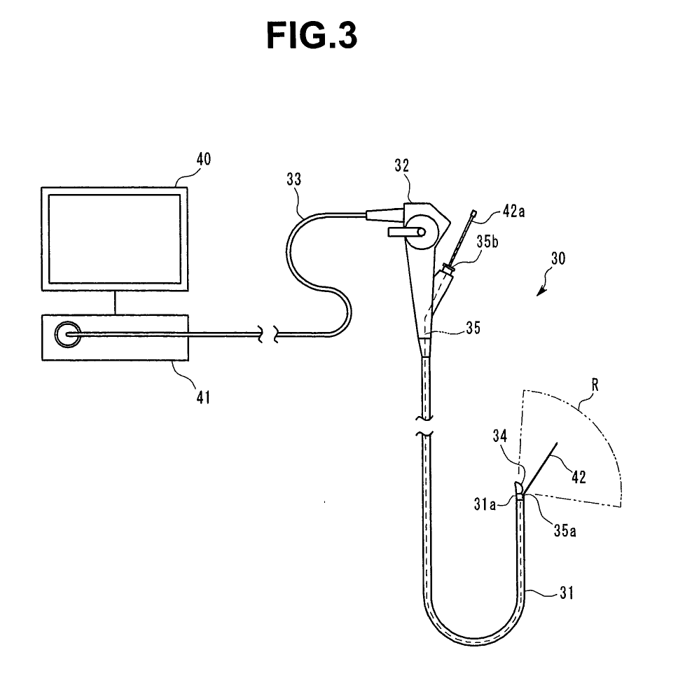

In the method of transurethral local anesthesia, a flexible ultrasound endoscope 30 illustrated in FIG. 3 is used. The ultrasound endoscope 30 is flexible, that is, the part to be inserted into the subject to be examined 1 is configured to be bendable. The ultrasound endoscope 20 is configured by mainly including an insertion portion 31 which can be introduced from the external urethral orifice 2a into the urethra 2 of the subject to be examined 1, an operation portion 32 located at the proximal end of the insertion portion 31, and a cord portion 33 extended from the operation portion 32. The cord portion 33 is connected to a control device 41.

The ultrasound endoscope 30 includes an ultrasound transducer 34 at a distal end portion 31a of the insertion portion 31. The ultrasound transducer 34 is driven by the control device 41. The ultrasound transducer 34 is capable of performing ultrasound beam scanning for generating an ultrasound observation image of the inside of the subject to be examined 1. The ultrasound observation image is generated by the control device 41 to be displayed on an image display device 40.

For example, the ultrasound transducer 34 is generally called a convex type, and configured to electronically scan the ultrasound beam in a sector manner like a region R shown by the two-dot chain lines in the drawing. The ultrasound endoscope 30 is capable of displaying a B-mode image and a Doppler image as ultrasound observation images on the image display device 40.

Furthermore, though not shown, the ultrasound endoscope 30 includes an optical observation device for performing optical observation and an illumination device for illuminating the field of view of an optical observation portion and may be configured to be able to provide an optical observation image of the inside of the subject to be examined 1 to a surgeon. The optical observation image may be in a form visually recognized directly by the surgeon through an eyepiece provided to the operation portion 32 or may be in a form displayed as an image on the image display device 40.

Furthermore, the ultrasound endoscope 30 includes a treatment instrument insertion channel 35 as a conduit which opens at the distal end portion 31a of the insertion portion 31 and the operation portion 32. The treatment instrument insertion channel 35 communicates a treatment instrument introducing port 35b as an opening portion of the insertion portion 31 with a treatment instrument outlet 35a as an opening portion of the distal end portion 31a.

An injection needle 42 made of a hollow tube, for example, can be inserted into the treatment instrument insertion channel 35. The injection needle 42 is inserted into the treatment instrument introducing port 35b, and thereby an injection needle sheath 42a is led out from the treatment instrument outlet 35a provided at the distal end portion 31a of the insertion portion 31. The injection needle sheath 42a can be connected also with a syringe used to manually leading out an anesthetic drug, though not shown.

Since the flexible ultrasound endoscope 30 as described above is publicly known, detailed description on the configuration thereof will be omitted.

In the method of transurethral local anesthesia, at first in a step S01, a mucosa anesthetic agent is injected from the external urethral orifice 2a into the urethra 2 using a syringe, not shown. The mucosa anesthetic agent is lidocaine jelly, for example. It is possible to numb the mucosa of the urethra 2 by injecting the mucosa anesthetic agent into the urethra 2, and dull the pain to be felt by the subject to be examined in a step, to be described later, in which the insertion portion 31 of the ultrasound endoscope 30 is inserted into the urethra 2.

Note that, if the mucosa anesthetic agent injected into the urethra 2 in the step S01 is a high lubricative jelly-like agent, the mucosa anesthetic agent works also as a lubricant to provide smooth lubrication between the urethral mucosa and the insertion portion 31 in a step, to be described later, in which the ultrasound endoscope 30 is inserted into the urethra 2.

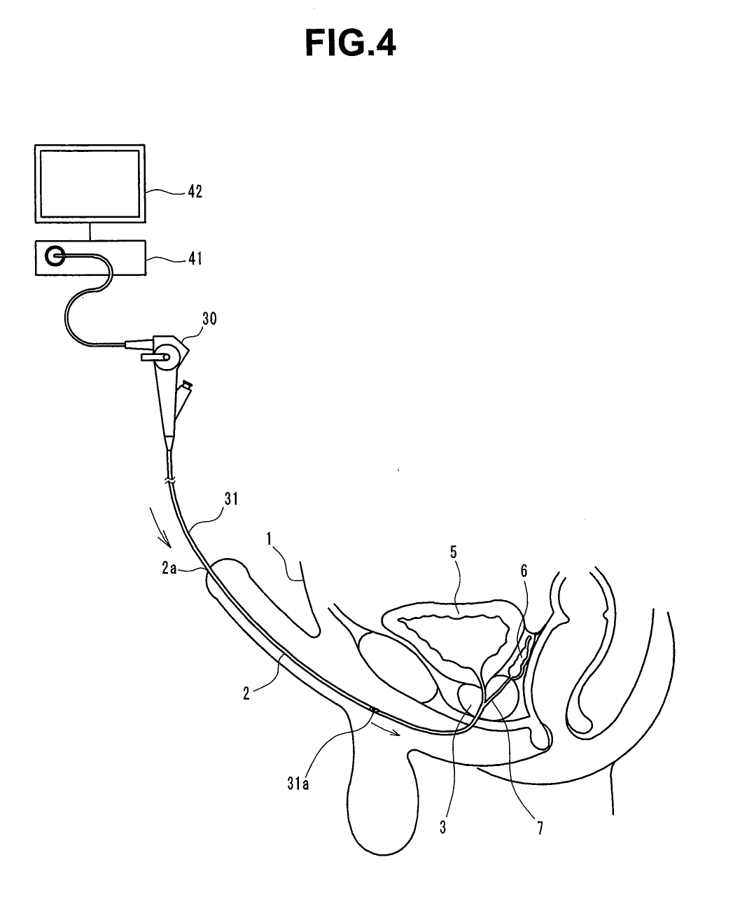

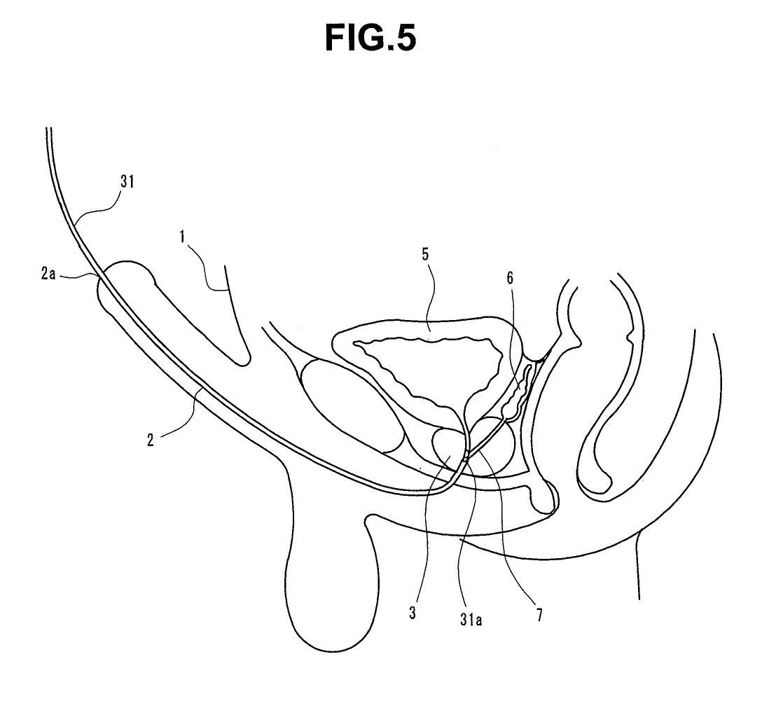

Next, in a step S02, the insertion portion 31 of the flexible ultrasound endoscope 30 is inserted from the external urethral orifice 2a into the urethra 2, as shown in FIG. 4. In the step S02, as shown in FIG. 5, the insertion portion 31 is inserted until the distal end portion 31a reaches the position where the prostate gland 3 exists.

In a case where the ultrasound endoscope 30 is provided with the optical observation device, if the insertion portion 31 is advanced to the position at which the junction between the urethra 2 and the ejaculatory duct 7 is observed on an optical observation image, the distal end portion 31 a reaches the position at which the prostate gland 3 exists.

Note that a method for checking whether or not the distal end portion 31a reaches the position at which the prostate gland 3 exists is not specifically limited. It is possible to check by using an ultrasound observation image obtained by the ultrasound transducer 34, or using an ultrasound observation image obtained by the ultrasound observation apparatus disposed on the body surface or in the rectum 4 of the subject to be examined 1.

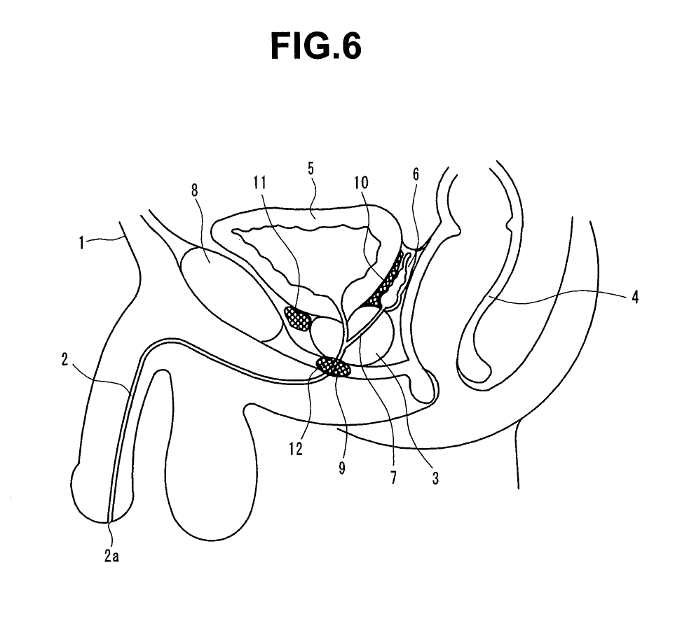

Next, in a step S03, the ultrasound transducer 34 of the ultrasound endoscope 30 is operated, and a position of a region to be anesthetized is identified while referring to the ultrasound observation image displayed on the image display device 40. Here, the region to be anesthetized is at least one of the three regions, approximate positions of which are indicated by the hatchings in FIG. 6.

In a first region to be anesthetized 10, a nerve bundle and a pelvic plexus located around the back side (right side in FIG. 6) and the upper side of the prostate gland 3 exist. The first anesthesia region 10 is a region surrounded by the prostate gland 3, the urinary bladder 5, and seminal vesicles 6. Note that FIG. 6 illustrates the cross section of the seminal vesicles 6. However, the seminal vesicles 6 are located separately on the left side and the right side at the upper position on the back side of the prostate gland 3.

In the second region to be anesthetized 11, a nerve bundle located around the belly side (left side in FIG. 6) of the prostate gland 3 exists. The second anesthesia region 11 is a region surrounded by the prostate gland 3, the urinary bladder 5, and a pubic bone 8.

In a third region to be anesthetized 12, an external urethral sphincter 9 and a nerve bundle leading to the external urethral sphincter 9 exist. The third region to be anesthetized 12 is a region including the external urethral sphincter 9 and the circumference of the external urethral sphincter 9.

In the method of transurethral local anesthesia according to the present invention, an anesthetic drug is injected into at least one of the first to third regions to be anesthetized 10, 11 and 12. In the step S03, as a preparatory step of injecting the anesthetic drug into the region to be anesthetized, the position of the region to be anesthetized is identified using at least one of the B-mode image and the Doppler image as the ultrasound observation images. In the step S03, the region to be anesthetized may be identified by using only the B-mode image, or by using the Doppler image in combination with the B-mode image. Blood vessels run side by side with the nerve bundle or the nerve plexus, for example. Therefore, finding the blood vessels using the Doppler image is helpful for identifying the region to be anesthetized in which the nerve bundle and the nerve plexus exist.

Note that, an optical observation image may be used in addition to the ultrasound observation image in the step S03. The junction between the urethra 2 and the ejaculatory duct 7 is located on the back side of the urethra 2. Therefore, it is possible to easily check in which direction the ultrasound observation image is directed by identifying the junction on the optical observation image. For example, the second region to be anesthetized 11 exists in a direction of about 180 degrees from the ejaculatory duct 7 around the circumferential direction of the urethra 2.

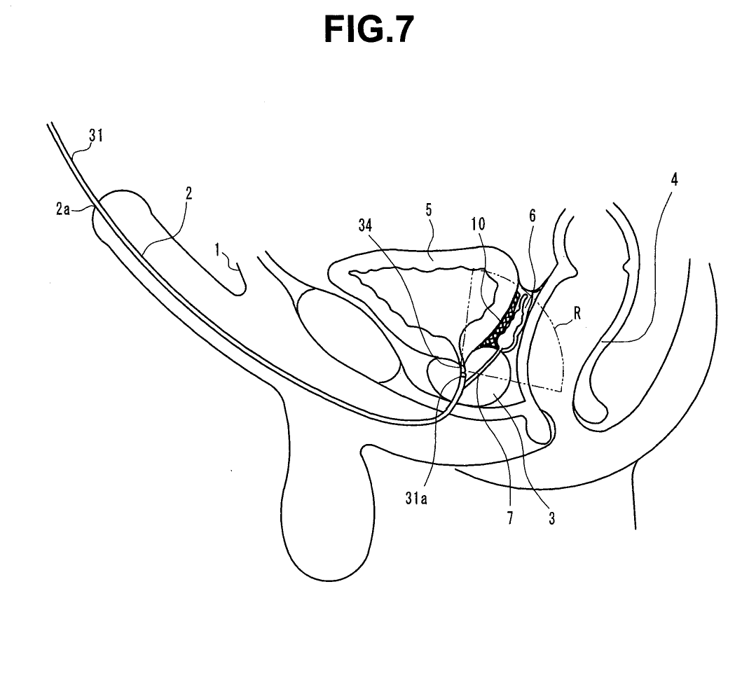

As one example in the case where the step S03 has been performed, FIG. 7 shows a state where the first anesthesia region 10 is set within a scanning region R of the ultrasound transducer 34. In the state shown in FIG. 7, the first anesthesia region 10 can be identified by using the ultrasound observation image.

Next, in a step S04, under the observation using the ultrasound observation image obtained by the ultrasound endoscope 30, the injection needle 42 is led out from the treatment instrument channel 35 of the ultrasound endoscope 30 and the anesthetic drug is injected into the region to be anesthetized identified in the step S03. Here, the anesthetic drug is injected into a plurality of sites in the region to be anesthetized identified in the step S03. Note that it is preferable to use the injection needle 42 whose gauge is equal to or smaller than 21G.

The anesthetic drug to be injected in the step S04 differs for each of the regions to be anesthetized. Specific examples thereof will be described below.

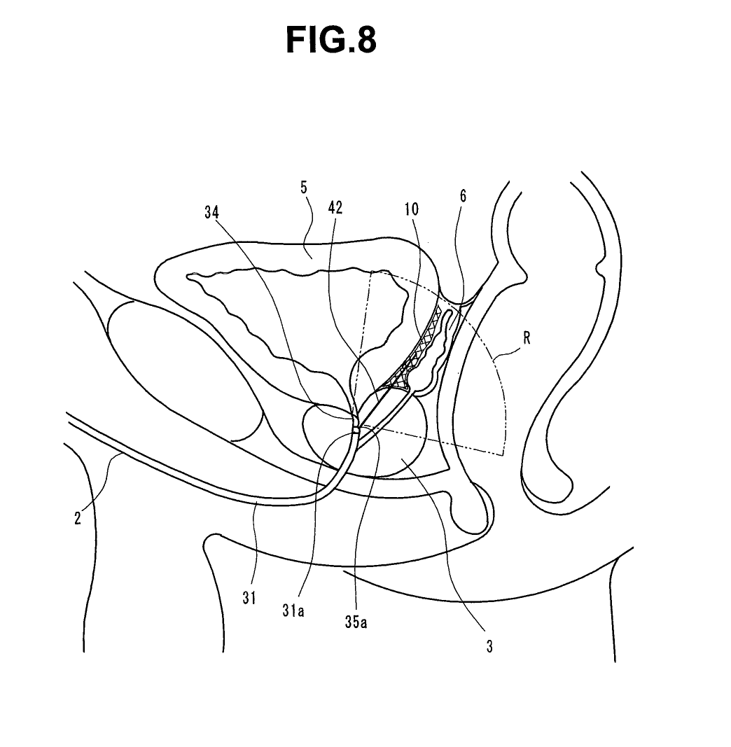

When the region to be anesthetized identified in the step S03 is the first region to be anesthetized 10, the injection needle 42 is punctured into the first region to be anesthetized 10 in the step S04, as shown in FIG. 8. As described above, the injection needle 42 is inserted into the treatment instrument insertion channel 35 and led out from the treatment instrument outlet 35a which opens at the distal end portion 31a of the insertion portion 31.

Then, a sensory nerve blocking agent as an anesthetic drug is injected into the first region to be anesthetized 10 through the injection needle 42. The sensory nerve blocking agent is lidocaine, mepivacaine, or the like, for example. The sensory nerve blocking agent is injected into a plurality of sites while changing the puncturing site of the injection needle 42 such that the sensory nerve blocking agent spreads over the whole of the first region to be anesthetized 10. According to this action, the sensory nerve blocking agent is injected into the nerve bundle and the pelvic plexus located around the back side and the upper side of the prostate gland 3.

By injecting the sensory nerve blocking agent into the first region to be anesthetized 10, the sensory nerves passing around the prostate gland 3 and extending toward the penis can be blocked. Therefore, it is possible to dull the pain felt by the subject to be examined 1 due to the insertion of the treatment instrument into the urethra 2.

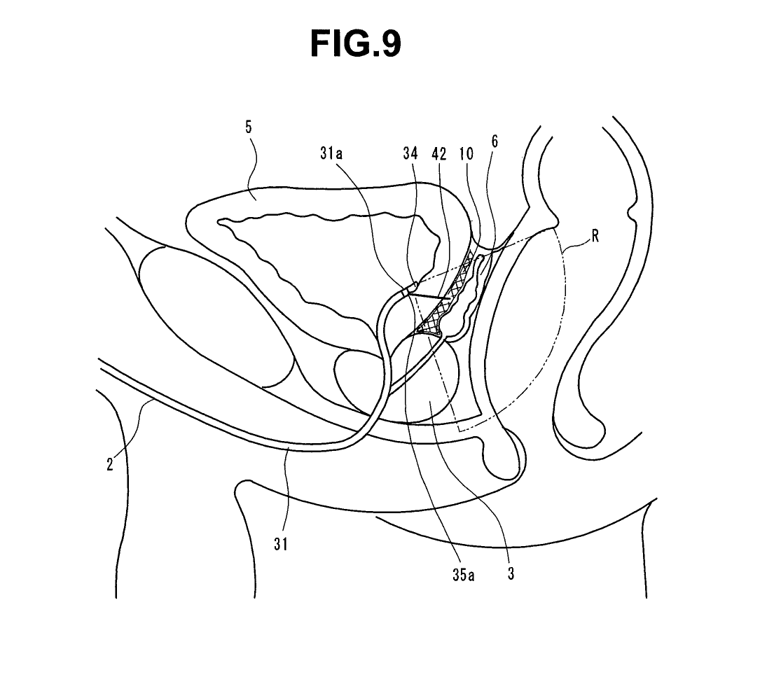

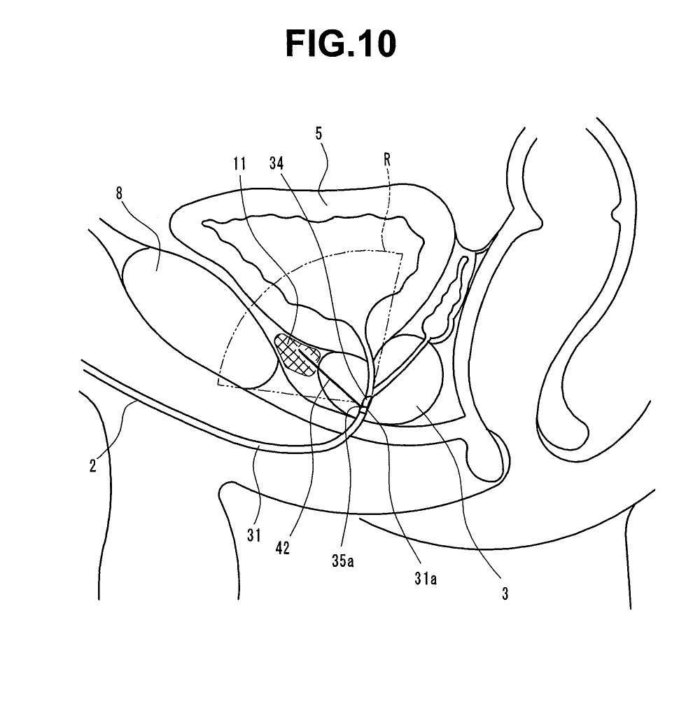

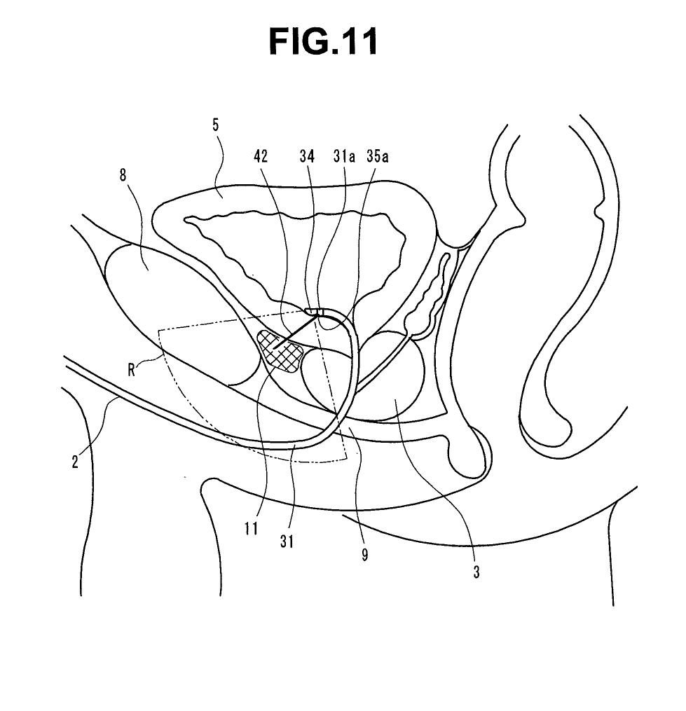

Note that the injection needle 42 is brought to reach the first anesthesia region 10 through the prostate gland 3 in FIG. 8. However, as shown in FIG. 9, the injection needle 42 may be brought to reach the first anesthesia region 10 from inside the urinary bladder 5. For example, when anesthesia is administered before making a diagnosis or perform therapy of the prostate cancer, for example, the distal end portion 31a of the insertion portion 31 of the ultrasound endoscope 30 is inserted into the inside of the urinary bladder 5, and the injection needle is punctured from inside the urinary bladder 5 into the first region to be anesthetized under the observation using an ultrasound observation image. The injection needle 42 is thus punctured from inside the urinary bladder 5 into the first anesthesia region 10, thereby capable of reducing a possibility of dissemination. At this time, when identifying the positions of the nerve bundle and the pelvic plexus into which the sensory nerve blocking agent is injected by using the ultrasound observation image, it is helpful to find blood vessels using the Doppler image, as described above. When the region to be anesthetized identified in the step S03 is the second region to be anesthetized 11, the injection needle 42 is punctured into the second region to be anesthetized 11 in the step S04, as shown in FIG. 10 or FIG. 11. Then, the sensory nerve blocking agent as the anesthetic drug is injected into the second region to be anesthetized 11 through the injection needle 42. The sensory nerve blocking agent includes lidocaine, mepivacaine, or the like, for example. The sensory nerve blocking agent is injected into a plurality of sites while changing the puncturing site of the injection needle 42 such that the sensory nerve blocking agent spreads over the whole of the second region to be anesthetized 11. According to this action, the sensory nerve blocking agent is injected into the nerve bundle located around the belly side of the prostate gland 3.

In the example shown in FIG. 10, the injection needle 42 is punctured into the second anesthesia region 11 through the prostate gland 3, and in the example shown in FIG. 11, the injection needle 42 is punctured into the second anesthesia region 11 from inside the urinary bladder 5.

By injecting the sensory nerve blocking agent into the second region to be anesthetized 11, the sensory nerves passing around the prostate gland 3 and extending toward the penis can be blocked. Therefore, it is possible to dull the pain felt by the subject to be examined 1 due to the insertion of the treatment instrument into the urethra 2.

Note that, when identifying the positions of the nerve bundle and the pelvic plexus into which the sensory nerve blocking agent is injected by using the ultrasound observation image, it is helpful to find blood vessels using the Doppler image, as described above. For example, when anesthesia is administered before making a diagnosis or performing therapy of the prostate cancer, for example, the injection needle 42 is punctured into the second anesthesia region 11 from inside the urinary bladder 5 without passing through the prostate gland 3, as shown in FIG. 11, thereby capable of reducing a possibility of dissemination.

When the region to be anesthetized identified in the step S03 is the third region to be anesthetized 12, the injection needle 42 is punctured into the third region to be anesthetized 11 in the step S04. Then, a muscle relaxant agent as an anesthetic drug is injected into the third region to be anesthetized 12 through the injection needle 42.

The muscle relaxant agent includes vecuronium bromide, pancuronium bromide, or the like, for example. The external urethral sphincter 9 included in the third region to be anesthetized 12 exists around the urethra 2. However the nerves exist on the back side. Therefore, the muscle relaxant agent is injected into a plurality of sites while changing the puncturing site of the injection needle 42 such that the muscle relaxant agent spreads over the region on the back side of the external urethral sphincter 9. Alternatively, the muscle relaxant agent may be injected into a plurality of sites while changing the puncturing site of the injection needle 42 by rotating the insertion portion 31 around an axis in the urethra 2 such that the muscle relaxant agent spreads over the whole of the external urethral sphincter 9.

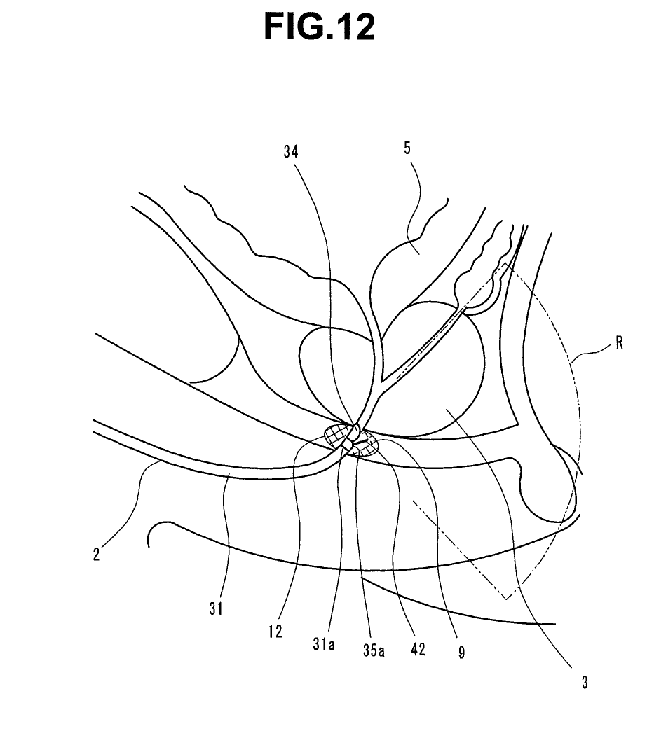

As shown in FIG. 12, by injecting the muscle relaxant agent into the third region to be anesthetized 12, it is possible to dull the pain felt by the subject to be examined 1 due to the external urethral sphincter being expanded by the treatment instrument inserted into the urethra 2.

In addition, as a modified example in the case where the region to be anesthetized identified in the step S03 is the third region to be anesthetized 12, a sensory nerve blocking agent may be injected as an anesthetic drug in addition to the above-described muscle relaxant agent. The sensory nerve blocking agent is lidocaine, mepivacaine, or the like, for example.

In this modified example, the sensory nerve blocking agent is injected into a plurality of sites while changing the puncturing site of the injection needle 42 such that the sensory nerve blocking agent spreads over the whole of the third region to be anesthetized 12. According to this action, the sensory nerve blocking agent is injected into the nerve bundle leading to the external urethral sphincter 9 and the nerve bundle located around the external urethral sphincter 9. In this modified example, the sensory nerve blocking agent may be injected before or after the injection of the muscle relaxant agent. According to the modified example, it is possible to dull the pain felt by the subject to be examined 1 due to the external urethral sphincter being expanded by the treatment instrument inserted into the urethra 2.

Note that a medicinal agent such as hyaluronan having relatively high viscous property may be added to the anesthetic drug to be injected in the step S04 as described above. It is more preferable to add such a medical agent, since it is possible to identify the site into which the anesthetic drug was injected by using the ultrasound observation image and duration of the anesthetic effect can be prolonged in this case.

Next, in a step S05, it is determined whether or not the anesthetic drug is injected into another region to be anesthetized different from the region to be anesthetized into which the anesthetic drug was injected in the already performed step S04.

In a case where the anesthetic drug is injected also into the other region to be anesthetized, in addition to the region to be anesthetized into which the anesthetic drug was already injected, the process returns to the step S03 and the other region to be anesthetized is identified using the ultrasound observation image, and then the anesthetic drug is injected into the other region to be anesthetized in the step S04.

That is, in the method of transurethral local anesthesia according to the present invention, the anesthetic drug may be injected into only one of the first to third regions to be anesthetized 10, 11 and 13, into a combination of any two of the first to third regions to be anesthetized 10, 11 and 13, or into all of the first to third regions to be anesthetized 10, 11 and 13.

In the step 05, when it is determined that there is no region to be anesthetized into which the anesthetic drug is injected, the process goes to a step S06, and the insertion portion 31 of the ultrasound endoscope 30 is extracted from the urethra 2.

Next, the process goes to step S07, and the endoscope/or the treatment instrument is inserted into the urethra 2 from the external urethral orifice 2a and the transurethral treatment is performed in a step S08. As described above, although the contents of the transurethral treatment are not specifically limited, in the present embodiment, the rigid endoscope 20 is inserted into the urethra 2 from the external urethral orifice 2a and prostatic biopsy treatment for harvesting the tissue of the prostate gland 3 is performed, as an example (FIG. 2). After the transurethral treatment is performed in the step S08, the process goes to a step S09, and the treatment instrument is extracted from the urethra 2. The injection needle 42 used when the anesthetic drug is injected in the step S04 has a diameter smaller than that of the needle for biopsy or that of the treatment instrument used in the step S07.

As described above, in the method of transurethral local anesthesia according to the present invention, local anesthesia is applied before performing the transurethral treatment on the prostate gland, the urinary bladder, the urethra, or the like. In the method, the local anesthesia is performed using the flexible ultrasound endoscope 30 which is inserted from the external urethral orifice 2a into the urethra 2. Therefore, in the present invention, the anesthesia required for performing the transurethral treatment can be performed without giving a pain to the subject to be examined 1. Thus, according to the present invention, there is no need for applying lumbar anesthesia when performing the transurethral treatment, thereby enabling the transurethral treatment to be quickly performed at a low cost.

The present invention is not limited to the above-described embodiment but various changes and modifications thereof are possible without departing from the gist of the invention.

What is claimed is:

1. A method of transurethral local anesthesia comprising steps of:
a step I of inserting a flexible ultrasound endoscope having a treatment instrument channel into a urethra or a urinary bladder;
a step II of identifying a region to be anesthetized by ultrasound observation using the ultrasound endoscope;
a step III of leading an injection needle out from the treatment instrument channel of the ultrasound endoscope and injecting an anesthetic drug into the region to be anesthetized while performing ultrasound observation using the ultrasound endoscope;
a step IV of extracting the ultrasound endoscope from the urethra; and
a step V of inserting, after the step IV, an endoscope and/or a treatment instrument into the urethra or the urinary bladder and performing transurethral treatment.

2. The method of transurethral local anesthesia according to claim 1, wherein the region to be anesthetized is at least one of a region surrounded by a prostate gland, the urinary bladder, and seminal vesicles and a region surrounded by the prostate gland, the urinary bladder, and a pubic bone in the step II.

3. The method of transurethral local anesthesia according to claim 2, wherein the anesthetic drug is a sensory nerve blocking agent in the step III.

4. The method of transurethral local anesthesia according to claim 1, wherein the region to be anesthetized includes an external urethral sphincter in the step II.

5. The method of transurethral local anesthesia according to claim 4, wherein the anesthetic drug is a muscle relaxant agent in the step III.

6. The method of transurethral local anesthesia according to claim 4, wherein the anesthetic drug is the muscle relaxant agent and the sensory nerve blocking agent in the step III.

7. The method of transurethral local anesthesia according to claim 4, wherein the region to be anesthetized further includes at least one of a region surrounded by a prostate gland, the urinary bladder, and seminal vesicles and a region surrounded by the prostate gland, the urinary bladder, and a pubic bone in the step III.
